(19) United States
(12) Patent Application Publication
(10) Pub. No.: US 2017/0335316 A1
(43) Pub. Date: Nov. 23, 2017

(54) METHODS AND COMPOSITIONS FOR SCREENING MOLECULAR FUNCTION COMPRISING CHIMERIC MINIMOTIFS

(71) Applicant: The Board of Regents of the Nevada System of Higher Education on Behalf of The Univ. of Nevada,, Las Vegas, NV (US)

(72) Inventors: Martin R. Schiller, Henderson, NV (US); Christy L. Strong, Henderson, NV (US)

(21) Appl. No.: 15/521,154

(22) PCT Filed: Oct. 19, 2015
(86) PCT No.: PCT/US15/56247

§ 371 Date: Apr. 21, 2017

Related U.S. Application Data
(60) Provisional application No. 62/066,556, filed on Oct. 21, 2014.

(51) Int. Cl. C12N 15/10; C12Q 1/68

(52) U.S. Cl. CPC C12N 15/1086; C12N 15/1065; C12Q 1/6897

(57) ABSTRACT

Disclosed herein are novel compositions and methods for elucidating biological activity and detection of molecular function. The methods and compositions disclosed herein can comprise the use of one or more minimotifs and a minimotif database for integrating and coordinating orthogonal knowledge derived from a variety of technological endeavors to provide systemic models representing complex biological and molecular interactions ranging from individual cells to entire organisms. The methods and compositions disclosed herein can utilize information related to biometrics including protein/protein interaction, and gene/gene interaction for evaluating cellular functions and cellular mechanisms.

### CROSS-REFERENCE TO RELATED APPLICATIONS

This application claims benefit of U.S. Provisional Application No. 62/066,556, filed Oct. 21, 2014 and is hereby incorporated herein by reference in its entirety.

### REFERENCE TO SEQUENCE LISTING

The Sequence Listing submitted Oct. 19, 2015 as a text file named “37474_0002P1_Sequence_Listing.txt,” created on Oct. 19, 2015, and having a size of 2,032 bytes is hereby incorporated by reference pursuant to 37 C.F.R. §1.52(e)(5).

### TECHNICAL FIELD

This invention relates to the field of molecular biology and protein biology involving the identification and detection of molecular functions using chimeric minimotifs. This application also relates to the fields of investigating biological function such as protein/protein interaction, as well as gene/gene interaction for evaluating cellular functions and cellular mechanisms to understand aberrant and disease conditions in order to facilitate improved diagnosis, and in order to enable targeted therapeutic intervention.

### BACKGROUND OF THE INVENTION

Modern day technological advances have enabled the gathering of vast amounts of data, using methods such as high throughput assays, and modeling large networks of metabolites, transcriptional responses, protein-protein interactions, and genetic interactions. Using such methods, large groups of data have been generated. Though useful, this data exists largely in discrete “entities” and until now, no convenient methodology has been available to integrate the knowledge based upon functional relationships and to make it available in a useful and practical format. Until now, techniques such as RNAi screens have been used to identify genes required for cell processes; this data may then be used to predict pathways and networks involved. However, molecular functions that mediate gene functions have not been sufficiently characterized. In effect, in most cases the “cause” (e.g. the gene or protein) has been identified, and the “effect” (e.g. function) has been identified too, what remains to be described is how the cause manifests into the function.

### SUMMARY

The present invention comprises novel methods and compositions for integrating and coordinating orthogonal knowledge derived from a variety of technological endeavors to provide systemic models representing complex biological and molecular interactions ranging from individual cells to entire organisms. Disclosed herein are unique methods comprising chimeric minimotif decoy technology for use in novel high throughput screens that enable the synergistic networking of information from other high throughput screens used in biological and biomedical sciences. The methods and compositions disclosed herein can comprise minimotifs, minimotif decoys, peptides, polypeptides, antibodies, nucleic acids, vectors, and host cells for making, using, assaying, and evaluating biological aspects of molecular and biological systems, including but not limited to, detecting molecular functions associated with diseased and aberrant metabolic states.

Disclosed herein are methods of preparing CMD clones comprising ligating a chimeric minimotif decoy initiator to a beginning end of minimotif duplex, ligating a chimeric minimotif decoy terminator to a terminal end of a minimotif duplex thereby forming a minimotif chimera cassette, ligating the minimotif chimera cassette to an expression vector, wherein the expression vector comprises a promoter and reporter protein under the control of the promoter, wherein the minimotif chimera cassette is ligated in frame with a reporter protein of the expression vector and expression of the chimeric protein containing the minimotifs is under the control of the promoter, vector, or cell permeant peptide vectors.

Disclosed herein are methods of preparing minimotif chimera cassettes or minimotif duplexes comprising synthesizing sense oligonucleotides comprising a linker region and a motif coding region, synthesizing antisense oligonucleotides comprising a linker region and a motif coding region, wherein the motif coding region of the antisense oligonucleotide is complementary to the motif coding region of the sense oligonucleotide, annealing the motif coding regions of the sense and antisense oligonucleotides, thereby forming a minimotif chimera cassette or minimotif duplex wherein the linker regions of the sense and antisense oligonucleotides remain single stranded.

Disclosed herein are methods of preparing minimotif chimeria cassette, comprising introducing a 5′ tagged chimeric minimotif decoy initiator to one or more minimotif oligonucleotides forming a first mixture, ligating a 5′ tagged chimeric minimotif decoy initiator to a beginning end of a minimotif oligonucleotide to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif oligonucleotide to form a 5′ and optionally 3′ tagged minimotif chimera cassette. The 5′ and optionally 3′ tagged minimotif chimera cassette can also be purified. In some embodiments, the purified 5′ and optionally 3′ tagged minimotif chimera cassettes can also be ligated with an oligonucleotide patch.

Disclosed herein are methods of preparing minimotif chimeria cassette, comprising introducing a 5′ tagged chimeric minimotif decoy initiator to one or more minimotif duplexes forming a first mixture, ligating a 5′ tagged chimeric minimotif decoy initiator to a beginning end of a minimotif duplex to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif duplex to form a 5′ and optionally 3′ tagged minimotif chimera cassette. The 5′ and optionally 3′ tagged minimotif chimera cassette can also be purified. In some embodiments, the purified 5′ and optionally 3′ tagged minimotif chimera cassettes can also be ligated with an oligonucleotide patch.

### BRIEF DESCRIPTION OF THE FIGURES

FIG. 1 provides a schematic depicting chimeric minimotif decoy (CMD) screening technology that identifies the roles of different molecular functions in assayable cell processes.

FIG. 2 provides a schematic showing CMD library design and construction. Synthetic minimotif duplexes encoding different minimotifs were randomly ligated with initiator and terminator duplex oligonucleotides to generate a plasmid expression library containing 1000s of CMD clones. Each clone had a SalI restriction site on the 5′ end and a BamHI site on the 3′ for subcloning into the pRSET.mCherry expression vector. This resulted in a plasmid library containing CMD clones with randomized minimotif composition and length. A DNA gel shows the size of the minimotifs inserts for 9 clones from CMD library #1. Inserts range in size from 1-9 minimotifs. The number of base pairs on the DNA ladder is indicated.

FIGS. 3A-3D show a CMD assay for HIV replication. FIGS. 3A-3D: GHOST cells expressing ectopic CD4 and CCR5 receptors are engineered to express GFP and fluoresce green upon HIV infection; GFP expression is under control of the HIV LTR which binds HIV Tat and drives transcription (FIG. 3A). FIG. 3B: GHOST cells infected with HIV and transfected with control empty pRSET-B.mcherry fluoresce both red and green. FIGS. 3C & 3D. When transfected with a CMD clone, these cells fluoresce red. The transfected clones are indicated in the bottom right of the panels. When challenged with HIV there are two possibilities. FIG. 3C. Cells fluorescing only red indicate that the CMD clone blocked HIV infection and is a positive hit. FIG. 3D. Cells fluorescing both red and green indicate that the CMD clone did not block HIV infection. This co-localization appears as an orange or yellow color. FIGS. 3A-3D Nuclei were stained with Hoescht. 50 CMD clones were screened producing 6 positive clones, variable subcellular localization (e.g. MM72 shows nuclear localization and MM16 and MM09 show Golgi localization), and 6 clones showed formation of HIV positive syncitia.

FIG. 4 provides a graphical depiction of Minimotif Miner (a minimotif database) highlighting the attributes and information contained related to individual minimotifs, including affinity, structure, references and experimental data.

FIG. 5 provides a schematic showing the process of designing the minimotifs in single stranded DNA oligonucleotide forms.

FIGS. 6A and 6B show a fluorescence screening assay. FIG. 6A provides a graphical depiction showing that infection by a functional HIV particle will cause subject cells to produce green fluorescent protein (GFP). FIG. 6B provides a schematic showing the basic premise of the fluorescence screen.

### DETAILED DESCRIPTION

### Definitions

The terminology used herein is for the purpose of describing particular aspects only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, an aspect includes from the one particular value and/or to the other particular value. Similarly, when values are expressed as approximations, by use of the antecedent “about,” it will be understood that the particular value forms an aspect. It will be further understood that the endpoints of each of the ranges are significant both in relation to the other endpoint, and independently of the other endpoint.

The amino acid abbreviations used herein are conventional three or one letter codes for the amino acids and are expressed as follows: Ala or A for Alanine; Arg or R for Arginine; Asn or N for Asparagine; Asp or D for Aspartic acid (Aspartate); Cys or C for Cysteine; Gln or Q for Glutamine; Glu or E for Glutamic acid (Glutamate); Gly or G for Glycine; His or H for Histidine; Ile or I for Isoleucine; Leu or L for Leucine; Lys or K for Lysine; Met or M for Methionine; Phe or F for Phenylalanine; Pro or P for Proline; Ser or S for Serine; Thr or T for Threonine; Trp or W for Tryptophan; Tyr or Y for Tyrosine; Val or V for Valine; Asx or B for Aspartic acid or Asparagine; and Glx or Z for Glutamine or Glutamic acid.

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. In addition, as used herein, the term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains, and the amino or carboxyl termini. The same type of modification can be present in the same or varying degrees at several sites in a given polypeptide.

As used herein, “cognate” refers to an entity of a same or a similar nature.

As used herein, the term “amino acid sequence” refers to a list of abbreviations, letters, characters, or words representing amino acid residues.

As used herein, “peptidomimetic” means a mimetic of a peptide, which includes some alteration of the normal peptide chemistry. Peptidomimetics typically enhance some property of the original peptide, such as increase stability, increased efficacy, enhanced delivery, increased half-life, etc. Methods of making peptidomimetics based upon a known polypeptide sequence are described, for example, in U.S. Pat. Nos. 5,631,280; 5,612,895; and 5,579,250. Use of peptidomimetics can involve the incorporation of a non-amino acid residue with non-amide linkages at a given position. One aspect of the present invention is a peptidomimetic wherein the compound has a bond, a peptide backbone or an amino acid component replaced with a suitable mimic. Some non-limiting examples of unnatural amino acids which may be suitable amino acid mimics include β-alanine, L-α-amino butyric acid, L-γ-amino butyric acid, L-α-amino isobutyric acid, L-ε-amino caproic acid, 7-amino heptanoic acid, L-aspartic acid, L-glutamic acid, N-ε-Boc-N-α-CBZ-L-lysine, N-ε-Boc-N-α-Fmoc-L-lysine, L-methionine sulfone, L-norleucine, L-norvaline, N-α-Boc-N-δCBZ-L-ornithine, N-δ-Boc-N-α-CBZ-L-ornithine, Boc-p-nitro-L-phenylalanine, Boc-hydroxyproline, and Boc-L-thioproline.

The word “or” as used herein means any one member of a particular list and also includes any combination of members of that list.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

As used herein, “reverse analog” or “reverse sequence” refers to a peptide having the reverse amino acid sequence as another reference peptide. For example, if one peptide has the amino acid sequence ABCDE, its reverse analog or a peptide having its reverse sequence is as follows: EDCBA.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels.

“Modulate”, “modulating” and “modulation” as used herein mean a change in activity or function or number. The change may be an increase or a decrease, an enhancement or an inhibition of the activity, function, or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100 percent, or more, or any amount of promotion in between compared to native or control levels. In an aspect, the increase or promotion is 10-20, 20-30, 30-40, 40-50, 50-60, 60-70, 70-80, 80-90, or 90-100 percent as compared to native or control levels. In an aspect, the increase or promotion is 0-25, 25-50, 50-75, or 75-100 percent, or more, such as 200, 300, 500, or 1000 percent more as compared to native or control levels. In an aspect, the increase or promotion can be greater than 100 percent as compared to native or control levels, such as 100, 150, 200, 250, 300, 350, 400, 450, 500 percent or more as compared to the native or control levels.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

The term “sample” can refer to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

As used herein, the term “minimotif” is used to describe short contiguous peptide sequences or sequence patterns in proteins with known biological function. “Minimotifs” can play important roles in most cellular functions and proteins, and they are involved in almost every cellular process. “Minimotifs” can serve different functions, including, but not limited to: (1) encoding binding to other molecules, including proteins, (2) locating covalent modification by enzymes, and (3) trafficking of proteins to specific cellular regions.

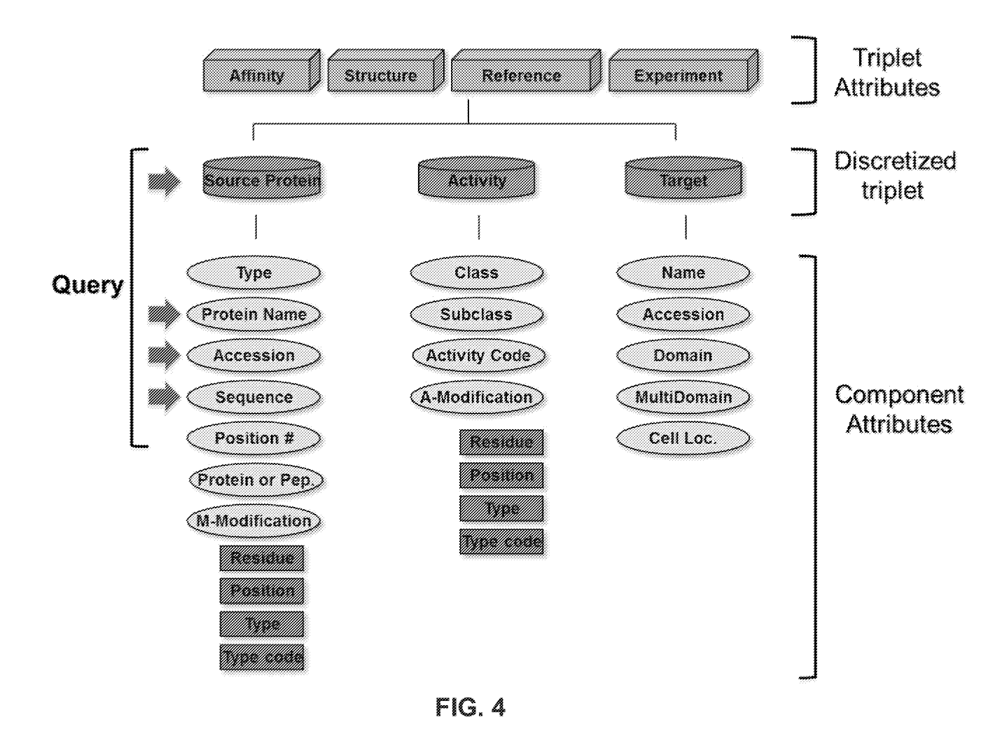

As used herein, the term “minimotif database” is used to describe a database or other sources of minimotif information wherein the molecular, cellular, and/or the biological functions of specific minimotifs are identified and described and linked with other attributes. Such attributes can be characterized by a syntactical quartet that includes information concerning the source protein of the minimotif, molecular activity, targets, and structure of the minimotif. The database can provide information including minimotif affinities, structure, minimotif modifications, references (e.g. published references), and experimental data. The source protein can be characterized by type (peptide/protein), protein name, accession data, sequence, position, and modification (residue, position, type, type code). Activity can be characterized by class, subclass, activity code, and modification (residue, position, type, type code). Minimotif targets can be characterized by name, accession, domain, multidomain, and cellular location. See FIG. 4.

As used herein, the term “minimotif chimera cassette” is used to describe a DNA sequence comprising three components: (1) a CMD initiator, (2) one or more minimotifs, and (3) a CMD terminator. Each of the three components consists of double stranded DNA. A CMD clone can be ligated into an expression vector in frame with a DNA sequence that encodes a label (e.g. a fluorescent fusion protein). For purposes of library construction, complementary oligonucleotide duplexes encoding minimotifs can be designed to encode a sticky-end overhang wherein the overhang can be 1-20, 4-18, or 4-10 nucleotides. Complementary oligonucleotides duplexes encoding minimotifs can be also be designed to include a linker (such as Gly-Ser) between the one or more minimotifs. In some embodiments, synthetic oligonucleotides may be phosphorylated with T4 polynucleotide kinase, annealed, and multiple minimotifs ligated together in the presence of initiator and terminator fragments. In some embodiments, minimotif chimera cassette as described herein can be ligated into a pRSET.mcherry vector

As used herein, the phrase “chimeric minimotif decoy initiator” is used to describe an oligonucleotide duplex that can be used in the preparation of a minimotif chimera cassette or a CMD clone. The chimeric minimotif decoy initiator can be used to ensure the minimotif chimera cassette, when ligated into an expression vector, is kept in frame with other sequences of the expression vector. For example, a chimeric minimotif decoy initiator can be used to ensure the minimotif chimera cassette, when ligated into an expression vector, is kept in frame with a reporter protein. In some aspects, the chimeric minimotif decoy initiator can be designed to encode a Kozak sequence, a start Methionine, and/or a restriction enzyme consensus sequence (e.g. a SalI cleavage site) on the 5′ end to facilitate subcloning a minimotif chimera cassette into a pRSET-mcherry vector.

As used herein, the term “chimeric minimotif decoy terminator” is used to describe an oligonucleotide duplex that can be used in the preparation of a minimotif chimera cassette or a CMD clone. A “chimeric minimotif decoy terminator” can optionally comprise a stop codon, a restriction enzyme consensus sequence for cloning into an expression vector, and/or an epitope tag(s). In some aspects, a chimeric minimotif decoy terminator may encode a myc epitope tag, stop codon, and BamHI cleavage site on the 3′ end for subcloning into the pRSET-mcherry vector.

As used herein, the term “Chimeric Minimotif Decoy (CMD) Library” is used to describe multiple CMD clones. Each clone comprises a minimotif chimera cassette (chimeric minimotif decoy initiator, one or more minimotifs, and a chimeric minimotif decoy terminator) ligated into an expression vector. The vector can be any vector, including, but not limited to: pRSET.mcherry, an expression vector such as pCDNA3.1, a fusion protein vector for bacterial expression (e.g. pGEX), a lentivector or adenoviral vector, or a vector for expression as a cell permeant peptide fusion.

As used herein, the term “linker region” is a DNA sequence capable of encoding amino acids that can occur between minimotif oligonucleotides, between minimotif duplexes, between chimeric minimotif decoy initiator and a minimotif duplex, between chimeric minimotif decoy terminator and minimotif duplex, between chimeric minimotif decoy initiator and minimotif oligonucleotide or between chimeric minimotif decoy terminator and minimotif oligonucleotide. As used herein, the term “linker region” can also refer to a DNA sequence capable of encoding amino acids that arise from ligation of or are created by ligating: (i) minimotif oligonucleotides, (ii) minimotif duplexes, (iii) a chimeric minimotif decoy initiator and a minimotif oligonucleotide, (iv) a chimeric minimotif decoy initiator and a minimotif duplex, (v) or a chimeric minimotif decoy terminator and a minimotif oligonucleotide, or (vi) a chimeric minimotif decoy terminator and a minimotif duplex A linker region can comprise DNA sequences that occur in increments of three base pairs (e.g. 3, 6, 9, 12, 15, etc.). For example, the linker regions can be used to join different minimotif oligonucleotides or duplexes within a minimotif chimera cassette. In some embodiments, a linker region that is capable of encoding two amino acids can be designed or ligated between one or more minimotif oligonucleotides or duplexes. Linker regions in single stranded DNA can also serve as hybridization partners for complementary single stranded DNA of linker regions of other synthetic oligonucleotide duplex minimotifs. In such embodiments, the linker regions can be designed to be complementary to each other.

As used herein, the term “minimotif oligonucleotide” describes a synthetic nucleic acid sequence that encodes a sense or antisense strand of a minimotif, and juxtaposed linker regions. Sense and antisense minimotif oligonucleotides that are complementary to one another can hybridize to one another to form minimotif duplexes that encode minimotif coding regions.

As used herein, the term “CMD clone” describes a vector (e.g. a plasmid or viral vector) that comprises a promoter and coding region for a chimera of (i) a chimeric minimotif decoy initiator, (ii) one or more minimotifs, minimotif chimeric oligonucleotides or minimotif duplexes, and, (iii) a chimeric minimotif decoy terminator. The CMD clones a can also comprise linkers. The CMD clone can also comprise an epitope tag and a label (e.g. a DNA sequence capable of encoding a fusion fluorescent protein).

As used herein, the term “motif coding region” describes a single or double stranded DNA sequence capable of encoding a minimotif sequence.

“Homology” refers to the resemblance or similarity between two sequences due to the organisms being of common ancestry (or descending from common evolutionary ancestor). Thus, two non-natural sequences are understood to not have an evolutionary relationship between the two and therefore instead of homology between non-natural sequences, similarity would be determined.

“Identity” is the degree of correspondence between two sub-sequences (no gaps between the sequences). For example, two nucleic acid sequences that have a certain number of nucleotides in common at aligned positions are said to be identical to that degree. An identity of 25% or higher can imply similarity of function, while 18-25% can imply similarity of structure or function.

Sequence “similarity” is the degree of resemblance between two sequences when they are compared. Similarity can be determined by the physic-chemical properties shared between those nucleotides at a certain position.

The term “subject” means any individual who is the target of administration. The subject can be a vertebrate, for example, a mammal. Thus, the subject can be a human. The term does not denote a particular age or sex. Thus, adult and newborn subjects, as well as fetuses, whether male or female, are intended to be covered. A patient refers to a subject afflicted with a disease or disorder.

The term “patient” includes human and veterinary subjects. Subject includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. The subject may to an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

### Methods and Compositions

Disclosed herein are methods and compositions for elucidating molecular function using chimeric minimotifs. The methods disclosed herein enable the evaluation of biological and molecular function including, but not limited to, protein/protein interaction, and gene/gene interaction. Use of chimeric minimotifs as described herein provides novel insight for evaluating cellular functions and cellular mechanisms in order to understand aberrant metabolic processes and disease conditions to facilitate improved diagnosis, and in order to enable targeted therapeutic intervention.

There continues to be an ongoing effort in science to understand “cells” and “whole organisms” (such as humans) as integrated systems by developing high throughput technologies and modeling large networks of metabolites, transcriptional responses, protein-protein interactions, genetic interactions, etc. Though large volumes of important information are gathered, most of these technologies create orthogonal knowledge, discrete pockets of data that need to be integrated in order to provide a systemic model of the cell and organism. Currently for example, a disconnect exists in the knowledge gained from high-throughput screens regarding protein function. RNAi screens are used to identify genes required for a cell process. These data are then used to predict the pathways and networks involved. However, until now, there has been no high throughput technology to experimentally identify the molecular functions that mediate gene interactions, which are commonly inferred in the system tested and not directly derived by experimentation.

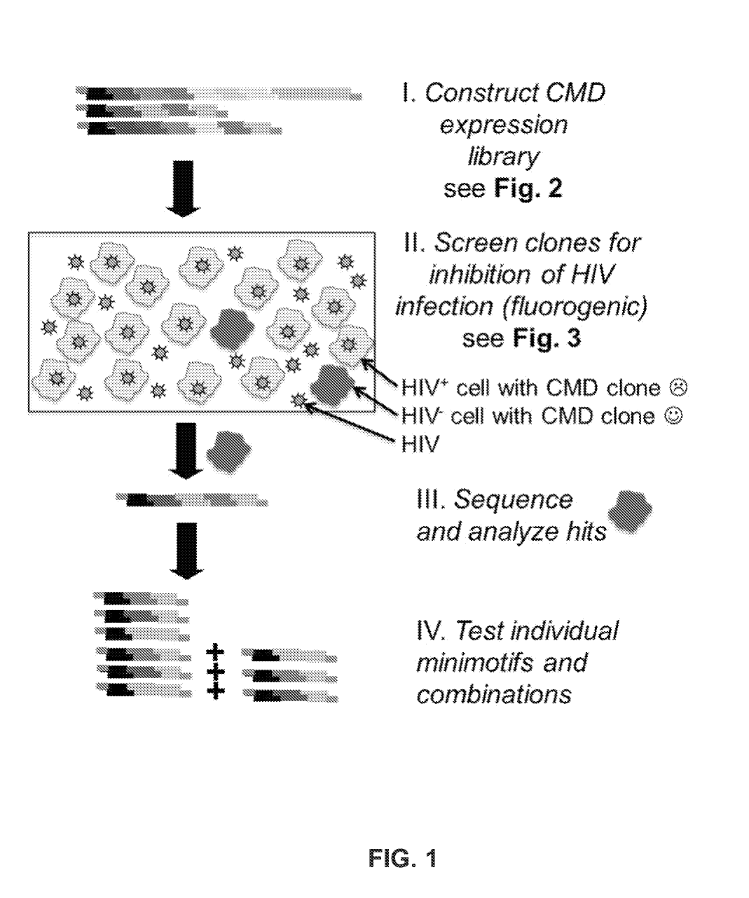

Disclosed herein are novel chimeric minimotif decoy (CMD) screening technologies that can be used to identify the roles of different molecular functions in assayable cell processes (FIG. 1). Disclosed herein are methods that can take advantage of minimotif databases. For example, the methods disclosed herein can take advantage of the information of a minimotif database or other sources of minimotif information. For example the Minimotif Miner database containing information about approximately 600,000 short functional peptide sequences with an experimentally determined molecular function can be used [1-3]. The methods disclosed herein can include the use of expression plasmid libraries generated from one or more minimotif chimera cassettes of random subsets of minimotifs appended in-frame to the end of a labeling DNA coding region such as one coding for red fluorescent protein. Individual clones can then be transfected into separated wells of a multi-well plate and scored in any type of high throughput assay. Positive clones can be sequenced and related back to the minimotif database to identify molecular functions involved in an assayed process.

Some of the method disclosed herein can be used as CMD screens. The methods disclosed herein can provide a unique approach that synergistically networks information from other high throughput screens used for discovery in biomedical sciences. Recent advancements in DNA sequencing technology now allow cost-effective sequencing of entire genomes. Genome Wide Association Studies (GWAS) have emerged as the method of choice to identify mutations present in a group of diseased individuals, when compared to healthy people [4]. One major challenge in applying this knowledge to health care is determining what these mutations do and which mutated genes are drugable. The CMD screens disclosed herein can provide an additional independent discovery approach to help address these problems.

The methods disclosed herein can be based upon, and leverages significant research on minimotifs. Minimotifs are short contiguous peptide sequences in proteins with a known biological function. Minimotif sequences encode numerous cellular functions including, but not limited to, binding to other molecules (including proteins), covalent modification by an enzyme, or trafficking of proteins to a specific cell region. The largest database of minimotifs in the world is Minimotif Miner (MnM) which now has >600,000 minimotifs [1-3]. Algorithms have been developed to accurately predict new minimotifs based on consensus sequences [1, 5-9] and have advanced the theoretical model of minimotifs [9, 10]. Minimotifs play important roles in most cellular proteins and are involved in almost every cell process. As described herein, the MnM database can be used to design libraries of chimeric minimotif decoy inhibitors that can be screened using the methods described herein as well as for interpreting the resulting sequences identified in the methods described herein.

In one aspect, the methods disclosed herein can be used to identify the roles of HIV and human genes and proteins in HIV infection (see e.g. Examples below). As shown herein, there are ˜2,400 host human proteins identified in HIV infection and replication called host dependency factors (HDFs)[11-17] However, even though HDFs were identified by multiple RNAi screens, there is little overlap in these genes identified by the independent screens. As provided herein, the methods described herein can be used to advance current knowledge about HDFs, HIV biology, and discover potential targets for therapeutic intervention. For example, the compositions and methods described herein can provide: (1) an independent approach to validate HIV infection host dependency factors (HDFs) identified by RNAi screens; (2) to identify the molecular basis of identified genetic interactions between some host dependency factors, thus providing an approach for a high throughput screen to identify molecular functions; (3) to identify novel host dependency factors which provide proof of principle for CMD as a discovery based screen; and (4) to identify combinations of different sets of minimotifs that, together block HIV infection. Such methods can be used to identify sets of drug targets that can be used for combinatorial drug therapy. As shown with HIV, the compositions and methods described herein can be applied to other aspects of society that involve a correlation between biological genotypes and phenotypes, such as other diseases, agricultural needs, ecological needs, diagnostics, genetic engineering, or transgenics. The compositions and methods described herein therefore provide an innovative approach for discovery of sets of targets that can be drugged concurrently. Many human health ailments are polygenic (involving many genes and pathways), a major problem for understanding disease etiology and for developing approaches for treating patients. The compositions and methods described herein can provide a unique approach that allows for the design of therapeutic intervention in aberrant states wherein more than one molecular function can be targeted.

Disclosed herein are methods of preparing a CMD clone comprising ligating a chimeric minimotif decoy initiator to a beginning end of minimotif duplex, ligating a chimeric minimotif decoy terminator to a terminal end of a minimotif duplex thereby forming a minimotif chimera cassette, ligating the minimotif chimera cassette into an expression vector, wherein the expression vector comprises a promoter and reporter protein under the control of the promoter, wherein the minimotif chimera cassette is ligated in frame with reporter protein of the expression vector and expression of the chimeric protein containing the minimotifs is under the control of the promoter, thereby preparing a CMD clone. In some aspects, the minimotif duplex comprises one or more minimotif coding regions. In some aspects, the minimotif duplex has a DNA sequence with a single strand overhang on the 5′ end of one strand that is complementary to a portion of a 3′ strand of a chimeric minimotif decoy initiator; wherein the minimotif duplex encodes a DNA sequence with a single strand overhang on the 3′ end of one strand that is complementary to a portion of a 5′ strand of a chimeric minimotif decoy terminator. In some aspects, the DNA overhang comprises overhangs of 3, 6, 9, 12, 15, 18, or 21 nucleotides. In some aspects, the DNA overhang on the 3′ end of each strand of the minimotif duplex or the 5′ end of the chimeric minimotif decoy terminator can be of different lengths and/or can encode one or more different amino acids. In some embodiments, the DNA overhang can encode a linker region that is capable of encoding one more amino acids that join one or more minimotifs within a minimotif duplex. In some aspects, the DNA overhang on the 5′ end of each strand of the minimotif duplex encodes a linker region that can be used to link together one or more minimotif duplexes or a minimotif duplex to a chimeric minimotif decoy initiator or a chimeric minimotif decoy terminator.

In some aspects, the chimeric minimotif decoy initiator can encode a Kozak sequence. In some aspects, the chimeric minimotif decoy initiator can comprise a start codon. In some aspects, the chimeric minimotif decoy initiator can encode a cleavage site on the 5′ end for subcloning a minimotif into an expression vector. For example, the chimeric minimotif decoy initiator can encode a restriction enzyme sequence (e.g. a SalI cleavage site). The restriction enzyme sequence can be a sequence that represents a cleavage site for any restriction enzyme. The cleavage site can be four, five, six, seven, eight, nine, ten, twelve, fourteen, sixteen or twenty nucleotides long. For example, the restriction enzyme sequence can be a cleavage site for any of the currently known restriction enzymes.

Vectors can be, but are not limited to pGEX6P for bacterial expression as a fusion protein, pET vector series for expression of just the minimotif chimera cassette in E. coli, and pCDNA3.1 for mammalian expression. Fluorescent vectors such as, but not limited to, pEGFP or pCMS can also be used. In some aspects, the expression vector can comprise pRSET-mcherry vector.

There are a number of additional compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems that can utilize plasmids, viral vectors, viral nucleic acids, phage nucleic acids, phages through the use of methods such as, electroporation, lipofection, calcium phosphate precipitation, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. Further, these methods can be used to target certain diseases and cell populations by using the targeting characteristics of the carrier.

Expression vectors can be any nucleotide construction used to deliver nucleic acids into cells (e.g., a plasmid), or as part of a general strategy to deliver nucleic acids, e.g., as part of recombinant retrovirus or adenovirus (Ram et al. Cancer Res. 53:83-88, (1993)). For example, disclosed herein are expression vectors comprising an one or more of the disclosed minimotifs.

The term “vector” is used to refer to a carrier molecule into which a nucleic acid sequence can be inserted for introduction into a cell. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al., 1989 and Ausubel et al., 1996, both incorporated herein by reference. Vectors can comprise targeting molecules. A targeting molecule is one that directs the desired nucleic acid to a particular organ, tissue, cell, or other location in a subject's body.

The term “expression vector” refers to a vector containing a nucleic acid sequence coding for at least part of a gene product capable of being transcribed. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described. There are a number of ways in which expression vectors may be introduced into cells. In certain embodiments of the invention, the expression vector comprises a virus or engineered vector derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kb of foreign genetic material but can be readily introduced in a variety of cell lines and laboratory animals (Nicolas and Rubenstein, 1988; Temin, 1986).

The retroviruses are a group of single-stranded RNA viruses characterized by an ability to convert their RNA to double-stranded DNA in infected cells; they can also be used as vectors. Other viral vectors may be employed as expression constructs in the present invention. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

Other suitable methods for nucleic acid delivery to effect expression of the disclosed compositions are believed to include virtually any method (viral and non-viral) by which a nucleic acid can be introduced into an organelle, a cell, a tissue or an organism, as described herein or as would be known to one of ordinary skill in the art. Such methods include, but are not limited to, direct delivery of nucleic acids such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

The expression vectors can include a nucleic acid sequence encoding a marker product. This marker product can be used to determine if the nucleic acid has been delivered to the cell and once delivered is being expressed. Preferred marker genes are the E. coli lacZ gene, which encodes β-galactosidase, and the gene encoding the green fluorescent protein.

As used herein, plasmid or viral vectors are agents that transport the disclosed nucleic acids, such as the minimotif chimera cassettes, minimotif oligonucleotides or minimotif duplexes into the cell without degradation and include a promoter yielding expression of the nucleic acid in the cells into which it is delivered. Viral vectors can be, for example, Lentivirus, Adenovirus, Adeno-associated virus, Herpes virus, Vaccinia virus, Polio virus, neuronal trophic virus, Sindbis and other RNA viruses. Also preferred are any viral families that share the properties of these viruses, which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason, are commonly used vectors. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of one to many genes depending on the size of each transcript. Positive or negative selectable markers can be included along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the shRNA is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. This type of vector can be the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter that directs cell-specific expression operably linked to a heterologous gene. Heterologous in this context refers to any nucleotide sequence or gene, which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed polynucleotides can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood, to a target organ, or inhaled into the respiratory tract to target cells of the respiratory tract. For example, a composition comprising a polynucleotide described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some aspects, a chimeric minimotif decoy terminator may be designed to be ligated onto the 3′ end of the section of one or more minimotif oligonucleotides or minimotif duplexes. In some aspects, the chimeric minimotif decoy terminator can encode a peptide tag. Peptide tags can include, but are not limited to, myc, flag, HA, 6HIS, GST, MBP, or Strep, CBP, Myc, V5, Fc, SpyTag and fluorescent tags such as but not limited to GFP tag.

A chimeric minimotif decoy terminator can comprise a stop codon. The chimeric minimotif decoy terminator can also comprise a restriction enzyme consensus sequence (e.g. a BamHI cleavage site) for subcloning into an expression vector. In some aspects, the expression vector can comprise a pRSET-mcherry vector, a fluorescent fusion protein, pCDNA3.1, a bacterial plasmid (e.g. pGEX), a lentivector, an adenoviral vector, or a cell permeant peptide vector.

Disclosed herein are methods of preparing annealed synthetic oligonucleotide complexes. In some aspects, annealed synthetic oligonucleotide complexes can be minimotif chimera cassettes or minimotif duplexes. For example, disclosed are methods of preparing annealed synthetic oligonucleotide complexes comprising: synthesizing a sense oligonucleotide comprising a linker region and a motif coding region, synthesizing an antisense oligonucleotide comprising a linker region and a motif coding region, wherein the motif coding region of the antisense oligonucleotide is complementary to the motif coding region of the sense oligonucleotide, annealing the motif coding regions of the sense and antisense oligonucleotides, thereby forming a duplex wherein the linker regions of the sense and antisense oligonucleotides remain single stranded. In some aspects, the oligonucleotide complex comprise overhangs on one or both ends of the synthetic oligonucleotide complex. In some aspects, the linker region of the sense oligonucleotide primer and the linker region of the antisense oligonucleotide primer are capable of hybridizing to one another. In some aspects, the linker region of the sense oligonucleotide can comprise a four to eight nucleotide overhang located at the 5′ end, and/or the antisense oligonucleotide can comprise a four to eight base nucleotide overhang located at the 3′ end. In some aspects, the linker region of the sense oligonucleotide may comprise GGTTCT, and/or the linker region of the antisense oligonucleotide can comprise AGAACC. In some aspects, the sense oligonucleotide and antisense oligonucleotides may be phosphorylated prior to hybridization. In some aspects, one or more additional minimotif oligonucleotides or minimotif chimera duplexes can be hybridized and/or ligated together to form a single minimotif duplex or minimotif chimera cassette. In some aspects, the linker region of the sense oligonucleotide of one synthetic minimotif duplex can be annealed to the linker region of the antisense oligonucleotide of a different synthetic minimotif duplex to form a minimotif chimera. In some aspects, minimotif chimera can further comprise a chimeric minimotif decoy initiator and/or a chimeric minimotif decoy terminator.

Disclosed herein are methods of preparing minimotif chimeria cassette, comprising introducing a 5′ tagged chimeric minimotif decoy initiator to one or more minimotif oligonucleotides forming a first mixture, ligating a 5′ tagged chimeric minimotif decoy initiator to a beginning end of a minimotif oligonucleotide to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif oligonucleotide to form a 5′ and optionally 3′ tagged minimotif chimera cassette. The 5′ and optionally 3′ tagged minimotif chimera cassette can also be purified. In some embodiments, the purified 5′ and optionally 3′ tagged minimotif chimera cassettes can also be ligated with an oligonucleotide patch.

Disclosed herein are methods of preparing minimotif chimeria cassette, comprising introducing a 5′ tagged chimeric minimotif decoy initiator to one or more minimotif duplexes forming a first mixture, ligating a 5′ tagged chimeric minimotif decoy initiator to a beginning end of a minimotif duplex to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif duplex to form a 5′ and optionally 3′ tagged minimotif chimera cassette. The 5′ and optionally 3′ tagged minimotif chimera cassette can also be purified. In some embodiments, the purified 5′ and optionally 3′ tagged minimotif chimera cassettes can also be ligated with an oligonucleotide patch.

Also, disclosed herein are methods for preparing a minimotif chimera cassette, comprising introducing a 5′ tagged chimeric minimotif decoy initiator to one or more minimotif oligonucelotides forming a first mixture, ligating the 5′ tagged chimeric minimotif decoy initiator to a beginning end of a minimotif oligonucleotide, to form a first 5′ tagged initiator minimotif chimera cassette, purifying the ligated complex using the 5′ tag of the 5′ tagged chimeric minimotif decoy initiator, ligating a 3′ tagged chimeric minimotif decoy terminator to the other end of the minimotif oligonucleotide to form a 5′ tagged initiator and 3′ tagged terminator minimotif chimera cassette, and purifying the minimotif chimera cassette using the 5′ or the 3′ tag of the minimotif chimera cassette. The 5′ tagged initiator and 3′ tagged terminator minimotif chimera cassette can be further ligated to an oligonucleotide patch to form a purified double-stranded 5′ tagged initiator and 3′ tagged terminator minimotif chimera cassette. The tags used in the methods described herein can be peptide tags, such as epitope tags. In some aspects, the 5′ tagged chimeric minimotif decoy initiator can form an internal duplex. In some aspects, the first mixture can be heated to separate an internal duplex of a 5′ tagged chimeric minimotif decoy initiator, while maintaining the duplex between both stands of the chimera. In some aspects, the first mixture can be cooled after one or more of the steps of the methods disclosed herein, to allow any unligated 5′ tagged chimeric minimotif decoy initiators to reform an internal duplex. In some aspects, the Tₘ of the internal duplex can be lower than the Tₘ of the one or more minimotif chimera/annealed synthetic oligonucleotide complexes.

Also, disclosed herein are methods for preparing a minimotif chimera cassette, comprising introducing a 5′ tagged chimeric minimotif decoy initiator to one or more minimotif duplexes forming a first mixture, ligating the 5′ tagged chimeric minimotif decoy initiator to a beginning end minimotif duplex, to form a first 5′ tagged initiator minimotif chimera cassette, purifying the ligated complex using the 5′ tag of the 5′ tagged chimeric minimotif decoy initiator, ligating a 3′ tagged chimeric minimotif decoy terminator to the other end of the minimotif duplex to form a 5′ tagged initiator and 3′ tagged terminator minimotif chimera cassette, and purifying the minimotif chimera cassette using the 5′ or the 3′ tag of the minimotif chimera cassette. The 5′ tagged initiator and 3′ tagged terminator minimotif chimera cassette can be further ligated to an oligonucleotide patch to form a purified double-stranded 5′ tagged initiator and 3′ tagged terminator minimotif chimera cassette. The tags used in the methods described herein can be peptide tags, such as epitope tags. In some aspects, the 5′ tagged chimeric minimotif decoy initiator can form an internal duplex. In some aspects, the first mixture can be heated to separate an internal duplex of a 5′ tagged chimeric minimotif decoy initiator, while maintaining the duplex between both stands of the chimera. In some aspects, the first mixture can be cooled after one or more of the steps of the methods disclosed herein, to allow any unligated 5′ tagged chimeric minimotif decoy initiators to reform an internal duplex. In some aspects, the Tₘ of the internal duplex can be lower than the Tₘ of the one or more minimotif chimera/annealed synthetic oligonucleotide complexes.

In some aspects, the purified ligated 5′ tagged initiator and 3′ tagged terminator minimotif chimera cassette can be fractionated by size. In some aspects, one or more of the purified ligated 5′ tagged initiator and 3′ tagged terminator minimotif chimera cassettes can be amplified (e.g via PCR) to produce inserts for ligation. In some aspects, the amplified purified inserts can be visualized to confirm DNA bands that can further be excised and further purified. Restriction digest followed by phenol/chloroform extraction and precipitation can also be performed on the purified inserts (e.g. SalI/BamHI) to prepare the inserts for ligation into an expression vector. In some aspects, the purified ligated 5′ tagged initiator and 3′ tagged terminator minimotif chimera cassettes can be inserted into an expression vector. In some aspects, the method can further comprise transforming an isolated clone into a cell (e.g. E. coli cells).

The minimotifs or polypeptides disclosed herein encompass naturally occurring or synthetic molecules, and may contain modified amino acids other than the 20 gene-encoded amino acids. The minimotifs and polypeptides described herein can be modified by either natural processes, such as post-translational processing, or by chemical modification techniques which are well known in the art. Modifications can occur anywhere in the disclosed minimotifs and polypeptides, including the backbone, the amino acid side-chains and the amino or carboxyl termini. The same type of modification can be present in the same or varying degrees at several sites in a given minimotif or polypeptide.

Disclosed herein are multimers of one or more polypeptides disclosed herein. In an aspect, a multimer comprises more than one of the monomers disclosed herein.

Modifications to the minimotifs or polypeptides can include, but are not limited to: acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation.

The minimotifs and polypeptides disclosed herein can have one or more types of modifications. Numerous variants or derivatives of the peptides and analogs of the invention are also contemplated. As used herein, the term “analog” is used interchangeably with “variant” and “derivative.” Variants and derivatives are well understood to those of skill in the art and can involve amino acid sequence modifications. Such amino acid sequence modifications typically fall into one or more of three classes: substitutional; insertional; or deletional variants. Insertions include amino and/or carboxyl terminal fusions as well as intrasequence insertions of single or multiple amino acid residues. Insertions ordinarily are smaller insertions than those of amino or carboxyl terminal fusions, for example, on the order of one to four residues. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once. Substitutions, deletions, insertions or any combination thereof may be combined to arrive at a final derivative or analog.

The polypeptides disclosed herein can comprise one or more substitutional variants, i.e., a polypeptide in which at least one residue has been removed and a different residue inserted in its place. Such substitutions generally are made in accordance with the table below and are referred to as conservative substitutions.

### Exemplary Conservative Amino Acid Substitutions

| Original | Exemplary Conservative |
|---|---|
| Residue | Substitutions |
| Ala | Ser |
| Arg | Gly, Gln |
| Asn | Gln; His |
| Asp | Glu |
| Cys | Ser |
| Gln | Asn, Lys |
| Glu | Asp |
| Gly | Ala |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function are made by selecting substitutions that are less conservative than those shown in the above Table, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions that are generally expected to produce the greatest changes in the protein properties are those in which: (a) the hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or hystidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, or (e) by increasing the number of sites for sulfation and/or glycosylation.

Polypeptides of the present invention are produced by any method known in the art. One method of producing the disclosed polypeptides is to link two or more amino acid residues, peptides or polypeptides together by protein chemistry techniques. For example, peptides or polypeptides are chemically synthesized using currently available laboratory equipment using either Fmoc (9-fluorenylmethyloxycarbonyl) or Boc (tert-butyloxycarbonoyl) chemistry. A peptide or polypeptide can be synthesized and not cleaved from its synthesis resin, whereas the other fragment of a peptide or protein can be synthesized and subsequently cleaved from the resin, thereby exposing a terminal group, which is functionally blocked on the other fragment. By peptide condensation reactions, these two fragments can be covalently joined via a peptide bond at their carboxyl and amino termini, respectively. Alternatively, the peptide or polypeptide is independently synthesized in vivo. Once isolated, these independent peptides or polypeptides may be linked to form a peptide or fragment thereof via similar peptide condensation reactions.

Those of skill in the art readily understand how to determine the sequence similarity between two or more proteins or two or more nucleic acids. For example, the similarity can be calculated after optimally aligning the two sequences. Another way of calculating sequence similarity can be performed by published algorithms. Optimal alignment of sequences for comparison may be conducted by the Smith-Waterman algorithm of Smith et al., 1981, by the Needleman-Wunsch algorithm of Needleman et al., 1970, by the search for similarity method of Pearson et al., 1988, by computerized implementations of these algorithms (GAP, BESTFIT, FASTA, and TFASTA in the Wisconsin Genetics Software Package, Genetics Computer Group, 575 Science Dr., Madison, Wis.), or by inspection.

Disclosed herein are methods and compositions including primers and probes, which are capable of interacting with the minimotifs, minimotif oligonucleotides, minimotif duplexes, minimotif chimera cassettes and polypeptides as disclosed herein. In certain embodiments the primers are used to support DNA amplification reactions. In certain embodiments primers comprise oligonucleotide sense or antisense strands. Primers can be used to amplify a sequence in a sequence specific manner, for example by PCR. Extension from a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR. It is understood that in certain embodiments, the primers can also be extended using non-enzymatic techniques, where for example, the nucleotides or oligonucleotides used to extend the primer are modified such that they will chemically react to extend the primer in a sequence specific manner. Typically the disclosed primers hybridize with complementary nucleic acids or region of the nucleic acids, or they hybridize with the complement of the nucleic acid or complement of a region of the nucleic acid.

The polynucleotides (primers or probes) can comprise the usual nucleotides consisting of a base moiety, a sugar moiety and a phosphate moiety, e.g., base moiety—adenine (A), cytosine (C), guanine (G), uracil (U), and thymine (T); sugar moiety—ribose or deoxyribose, and phosphate moiety—pentavalent phosphate. They can also comprise a nucleotide analog, which contains some type of modification to either the base, sugar, or phosphate moieties. Modifications to nucleotides are well known in the art and would include for example, 5 methylcytosine (5 me C), 5 hydroxymethyl cytosine, xanthine, hypoxanthine, and 2 aminoadenine as well as modifications at the sugar or phosphate moieties. The polynucleotides can contain nucleotide substitutes which are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

The size of the primers or probes for interaction with the minimotifs in certain embodiments can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The nucleic acids, such as the oligonucleotides to be used as primers, can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein and nucleic acid molecules can be made using known methods such as those described by Nielsen et al., Bioconjug. Chem. 5:3-7 (1994).

The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997), incorporated herein by reference.

Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include primers to perform the amplification reactions described, as well as the buffers and enzymes required to use the primers as intended. For example, disclosed is a kit for assessing the role of a gene or gene sequence in any assayable biological process. For example, disclosed are kits for assessing the role of a gene or gene sequence in a molecular or biochemical pathway. In some aspects, discussed are kits for assessing the role or a gene or gene sequence in drug resistance. The kit can include instructions for using the reagents described in the methods disclosed herein.

Also disclosed herein are methods for detecting the presence of biomarkers in bodily fluid samples from patients wherein the samples comprise circulating aberrant cells from patients with biological issues.

It will be appreciated by those skilled in the art that the disclosed minimotifs, minimotif duplexes, minimotif chimera cassettes, minimotif oligonucleotides, polypeptides, and nucleic acids as well as the polypeptide and nucleic acid sequences identified from any subject or patient can be stored, recorded, and manipulated on any medium that can be read and accessed by a computer. The disclosed methods can be performed in silico. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000, 5000, 10,000, or more minimotifs, polypeptides or nucleic acids of the invention or polypeptide sequences or nucleic acid sequences identified from any subject or patient.

Thus, provided herein is a computer system comprising a database including records for minimotifs and nucleic acids encoding minimotifs. Disclosed herein is a computer system comprising a database including records for minimotifs and nucleic acids comprising the sequences encoding variants of minimotifs. Computer readable medium include magnetically readable media, optically readable media, electronically readable media and magnetic/optical media. For example, the computer readable medium may be a hard disc, a floppy disc, a magnetic tape, CD-ROM, DVD, RAM, or ROM as well as other types of other media known to those skilled in the art.

Aspects of the present invention include systems, particularly computer systems that contain the sequence information described herein. As used herein, “a computer system” refers to the hardware components, software components, and data storage components used to store and/or analyze the nucleotide sequences of the present invention or other sequences. The computer system preferably includes the computer readable media described above, and a processor for accessing and manipulating the sequence data of the disclosed compositions including, but not limited to, the disclosed minimotifs, polypeptides, and nucleic acids.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In an aspect, the computer system includes a processor connected to a bus which is connected to a main memory, preferably implemented as RAM, and one or more data storage devices, such as a hard drive and/or other computer readable media having data recorded thereon. In an aspect, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In an aspect, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device. Software for accessing and processing the nucleotide sequences of the nucleic acids of the invention (such as search tools, compare tools, modeling tools, etc.) may reside in main memory during execution.

In an aspect, the computer system comprises a sequence comparer for comparing minimotif, polypeptide and nucleic acid sequences stored on a computer readable medium to another test sequence stored on a computer readable medium. A “sequence comparer” refers to one or more programs that are implemented on the computer system to compare a nucleotide sequence with other nucleotide sequences and to compare a polypeptide with other polypeptides.

Accordingly, an aspect of the present invention is a computer system comprising a processor, a data storage device having stored thereon a minimotif, polypeptide, or nucleic acid of the invention, a data storage device having retrievably stored thereon reference minimotif, polypeptide, or nucleotide sequences to be compared with test or sample sequences and a sequence comparer for conducting the comparison. The sequence comparer may indicate a homology level between the sequences compared or identify a difference between two or more sequences.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

In these principle experiments, CMD technology was used in testing fluorogenic HIV infection assays. A plasmid library containing minimotifs was built and screened to identify minimotif and minimotif combinations that are required for HIV infection. It was demonstrated that some minimotifs can be rediscovered as inhibiting HIV infection, providing proof of principle for this approach.

### Identifying HIV Infection Inhibitors in Proof of Principle Experiments.

HIV infection was studied as a model for proof of principle experiments validating the CMD approach because: (1) viruses use minimotifs, many of which are required to take over cells [18]; (2) HIV proteins have 218 known minimotifs, of which 27 are required for infection and/or replication [19-50]; (3) the T20 minimotif has been developed into a fusion inhibitor, called Enfurvirtide, that is approved by the FDA and currently used to treat patients infected with HIV [51]; and (4) established HIV high throughput infection assays have been adapted herein. Nevertheless, it is important to note that this technology can be used in any system where an expression vector can be introduced and screened with a high-throughput assay.

Viruses like HIV are not living and must infect cells to use the host machinery for replication. Scientists have used several RNAi screens to identify ˜2,400 host human proteins required for HIV replication, and thus required for at least one aspect of the viral life cycle [11-17]. RNAi screens have the advantage of identifying a human protein abducted by the virus, but do not determine how the virus uses the protein. The methods disclosed herein can be used synergistic with current genetic approaches by not only identifying the gene involved in HIV infection, but identifying the specific amino acids that are critical for a defined molecular function and the basics of the mechanism by which proteins work together. Thus, a CMD clone can identify sets of drug targets that could be targeted together or be used to build a network of molecular interactions used by HIV to take over cells.

### Construction of a CMD Library #1.

The Minimotif Miner database was searched for minimotifs in HIV proteins and identified ˜218 minimotifs. These minimotifs are also shown in the HIVToolbox website [52]. 27 of these minimotifs, when mutated in HIV, significantly blocked replication by HIV in cell culture assays, indicating that some can inhibit HIV replication when expressed separately as minimotif decoys [19-50].

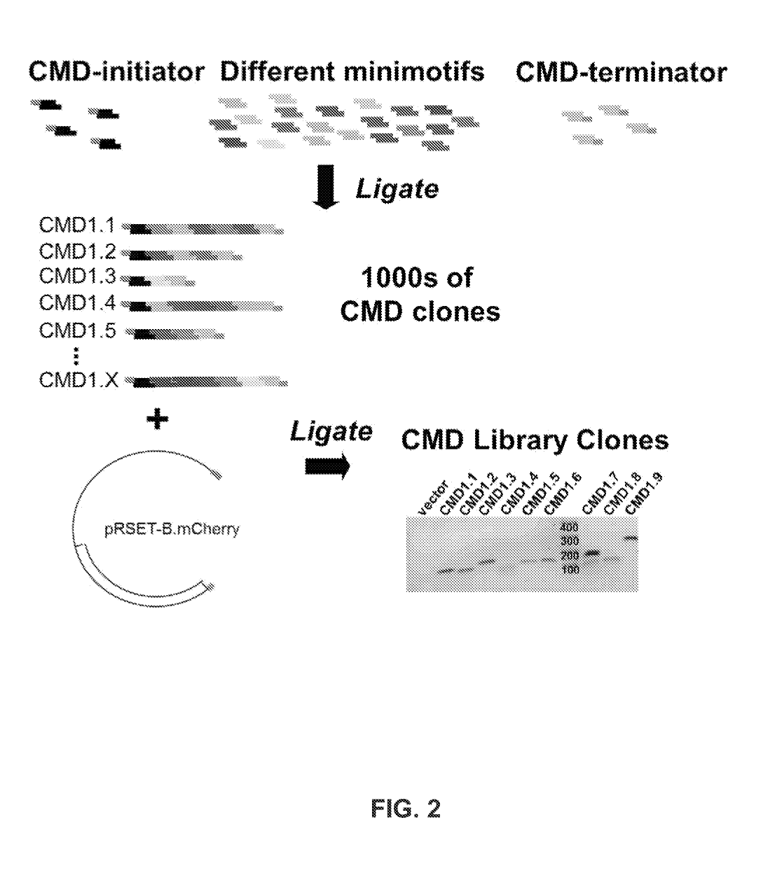

A DNA library of multiple random sets of 27 HIV minimotifs subcloned into vectors that express a red fluorescent protein with the minimotif chimera cassette fused to the C-terminus was built (FIG. 2). The library was built by random ligation of a mixture of the 27 minimotif duplexess encoding these minimotifs. These inserts were cloned into a plasmid expression library and characterized. CMD library #1 contains >10,000 clones. To evaluate the library, plasmid DNA was isolated for 37 clones and sequenced. The numbers of minimotifs in each clone had an average and mean of 3 minimotifs and ranged from (1-9 minimotifs) with no observed clone duplication and diverse representation of minimotifs.

### CMD Assay for HIV Replication.

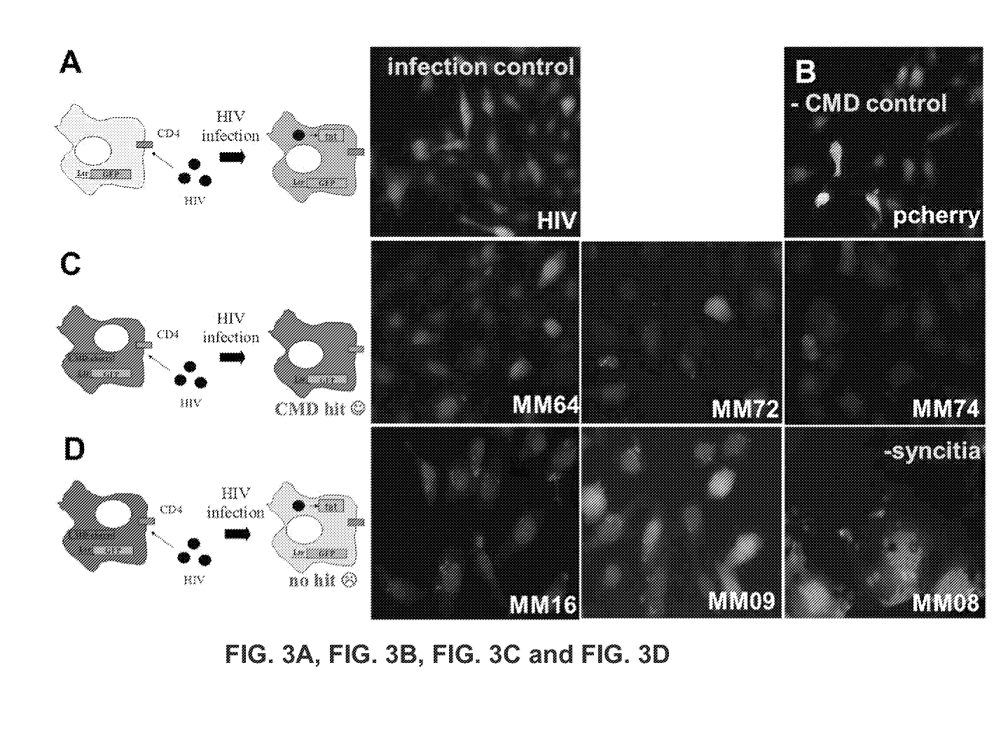

An assay was adapted by which CMD clones can be screened for the ability to inhibit HIV infection. (See methods). A HIV infection reporter cell line (GHOST cells) that, when infected with HIV, fluoresces green (FIG. 3) was used [53]. In the assay, a CMD inhibitor clone was transfected into the GHOST cells (by transfection) and those cells that were transfected fluoresced red. After 2 days, these cells were challenged with HIV for an additional day, and then analyzed by fluorescence microscopy. A high throughput 96 well plate format enabled rapid analysis of 1000s of individual CMD clones. Programmatic cell edge detection and quantification of the fluorescent signals using a Nikon software package were used to objectively identify CMD clones that inhibit HIV infection. (See methods). There were four possible outcomes: (1) cells transfected with a CMD clone, but not challenged with HIV fluoresced red; (2) cells that have been infected with HIV produced Green Fluorescent Protein (GFP) and fluoresced green; (3) cells that were transfected with a CMD clone and were infected when challenged with HIV fluoresced both green and red (FIG. 3; colored yellow); and (4) cells that were transfected with a CMD clone, challenged with HIV, and fluoresced only red, indicate that the CMD clone blocked HIV infection.

Rediscovery of Minimotifs that Block HIV Infection.

A preliminary test was performed screening 50 CMD clones and example results are shown in FIG. 3. Cells infected with HIV showed good induction of GFP expression, that was not observed in uninfected cells as expected (FIG. 3A). In cells transfected with empty pRSET.mcherry vector and infected with HIV, there were many cells fluorescing both green and red indicating transfection and infection of the same cells (FIG. 3B); the transfection efficiency was ˜38%, so some cells do not express the red fluorescent protein and fluoresce green upon infection. Similar results were observed when cells were transfected with 44 of the 50 CMD clones tested, indicating that these combinations of minimotifs do not block HIV infection (e.g. FIG. 3D, clones MM16, MM72, and MM74). Six of the 50 CMD clones tested showed either green or red cells (e.g. FIG. 3C, clone MM64, MM72, and MM74) indicating that these CDM clones blocked HIV infection. Two of the hits were retested for an extended time of inhibiting HIV replication. Both CMD clones showed reproducible inhibition of HIV infection for 1 day and infection was slowed, but some was apparent after 3 days.

Clones were conservatively only considered to be a positive hit when several hundred cells in 5 separate images were examined and a cell that fluoresced both red and green was never found. These clones contained 1-9 minimotifs. One clone (MM74) had a single minimotif for the interaction of GP41 with TIP47 and retrograde trafficking of the GP41 precursor, env [22]; a different minimotif for interaction with TIP47 was also identified in another positive hit (MM56). A second clone (MM72 had three minimotifs), one of which was for acetylation of the Tat transcriptional activator by PCAF, which is of interest as this clone was localized to the nucleus (FIG. 3C).

Single minimotif analyses are used to determine which of the minimotifs in each clone contribute to inhibition and this assay. Here each minimotif chimera cassette comprising only one type of minimotif was generated and then combinations of these motifs can be used to see which minimotifs in the original CMD clone were necessary for the activity.

There are several interesting observations about the CMD screen. Clones have different subcellular localization, which is dependent on the other minimotifs in the clone. For example, in FIG. 3 MM72 is nuclear, MM16 is in the Golgi region, and MM74 is cytoplasmic. 6 clones that induced formation of very large syncytia as shown for CMD clone MM08 in FIG. 3D were observed. While HIV induced syncytia formation is mediated by cell fusion where CD4+ cells fuse with cells expressing HIV GP41/GP120 [54], the screen used herein has the unexpected advantage that it identifies key molecular function involved in the cell fusion. Note that HIV infection in both transfected and untransfected cells are fused to form syncytia. As an aside, syncytia is not included in the assignment of positive or negative to a CMD clone because it cannot be determined whether the transfected cell was successfully infected first or just fused with a HIV infected cell [54].

Like a genetic screen, the demonstration of the CMD technology on HIV infection shows discovery of both suppressor and enhancer minimotifs in genes. Furthermore, the CMD technology has the advantages that it also identifies molecular functions and sets of genes that work synergistically as enhancers or suppressors in a high-throughput screen.

### Construction of a CMD Library #2.

In one aspect, a CMD screen was designed to discover novel minimotifs in host proteins that inhibit HIV infection or minimotif combinations that work together to inhibit HIV infection. The first library was stacked with minimotifs in HIV proteins that are required for HIV replication. Here, a new library comprised of minimotifs that more broadly cover different host proteins and functions in the human proteome was built. A second version of this library also contains known HIV HDFs.

Synthesized minimotif oligonucleotides were used to generate duplexes that encode ˜480 minimotifs from the ˜300,000 minimotifs for human proteins in the MnM 3.0 database [3]. These minimotifs were selected based on three criteria: (1) they differ in molecular activity (binds, modifies, traffics) and subactivity (e.g. phosphorylates, myristoylates, etc.); (2) they cover different cell processes by selecting from proteins with unique terms in the Gene Ontology database [57]; and (3) a subset includes the 2400 HIV HDFs. Other minimotifs include several negative controls (minimotifs in proteins with specialized cell function not relevant to HIV infection—e g minimotifs in thyroglobulin), the positive control minimotifs in CMD library #1.

Several different types of libraries are constructed for screening. The first library screened contains all minimotifs from libraries 1 and 2, which returns the positive clones identified in Library 1, and perhaps some minimotifs not known to play a role in HIV replication. Another library only has the HIV HDF minimotifs to provide both independent validation of HDFs and to identify the molecular basis for interaction between HDFs & HIV proteins. Another has no known positive or HDF minimotifs, which promotes discovery of novel minimotifs involved in HIV infection.

### Clone Validation.

Select minimotifs of interest identified in the CMD screen are validated. Selection is based on novelty and current knowledge about HIV cell biology. For these minimotifs, the sequences of the proteins that the minimotif is found in (source) and the target protein of the interaction are known. siRNAs to the minimotifs source and target proteins, alone and together, are used to confirm that one or both proteins are required for HIV infection. Western blot analysis is used to ensure that the protein levels are reduced in these experiments.

Synthetic DNAs are purchased, subcloned, expressed, and purified as GST-fusion proteins. One GST fusion protein is cleaved with thrombin to remove the GST portion, and purified so that binding can be evaluated. GST fusion proteins containing the minimotif appended to the C-termini are also generated. Site directed mutagenesis is used to convert the consensus amino acid positions to alanines. These experiments assess direct interactions and whether mutation of the minimotifs blocks the interactions. The synthetic DNA is also subcloned into an expression vector in frame with an epitope tag. These constructs are transfected into hEK-293 cells, then used for co-immunoprecipitation experiments to determine if the proteins interact in cells. Considering the amount of effort involved, this is only done for 1-3 clones to ensure that the CMD screen is identifying real interactions.

### Bioinformatic Analysis of CMD Results.

The lab has built many different types of bioinformatics applications, housed at bio-toolkit.com [7,52,58-61]. In one aspect, disclosed herein is a Java program that reads a file containing the sequence data from the CMD screen, pulls data from the Minimotif Miner database, and generates a report about what was identified in the screen. The report contains: (1) all minimotifs present in each clone and the order; (2) the frequency of minimotifs identified among all sequenced clones; (3) global statistics such as the average and range of minimotifs/clone; (4) data about the minimotifs—activity, target, Gene Ontology function, molecular pathway or process, etc.; and (5) anomalies in sequence of a clone. Other information may be included.

In the specific case of this HIV screen, the report contains information related to the HIV HDFs identified herein. This information is used to construct a network of HDFs that include molecular functions that are required for HIV infection. This helps validate HDFs identified by siRNA screens and also provides the molecular basis of interactions of different pairs of HDFs.

### Method

CMD Library Construction.

Complementary oligonucleotides encoding minimotifs were designed to encode a 6 nucleotide sticky-end overhang and for a Gly-Ser linker between minimotifs when ligated together. The chimeric minimotif decoy initiator was designed to be ligated onto the 5′ end, encode a Kozak sequence and start Methionine, and a SalI cleavage site on the 5′ end for subcloning into the pRSET-mcherry vector. The chimeric minimotif decoy terminator encodes a myc epitope tag, stop codon, and BamHI cleavage site on the 3′ end for subcloning into the pRSET-mcherry vector. Minimotif oligonucleotides were phosphorylated with T4 polynucleotide kinase, annealed, and multiple minimotifs were ligated together in the presence of chimeric minimotif decoy initiators and chimeric minimotif decoy terminators as described herein [62, 63]. This library was ligated into the pRSET.mcherry vector and transformed into E. coli (FIG. 2).

HIV Infection Assay.

GHOST (3) Hi-5 cells were provided by the NIH AIDS Research and Reference Reagent Program. These cells express CD4 and the CCR5 co-receptor for HIV entry and contain a HIV-2 LTR driven GFP reporter (FIG. 3) [53]. When these cells are infected with HIV, Tat binds to the LTR and drives the expression of GFP, which can readily be detected by fluorescence microscopy. This part of the assay assesses all steps of the viral life cycle up to the expression of Tat, but not expression of other proteins, construction, and secretion of HIV particles [13]. To assess these steps, after an initial infection period (to be optimized), media containing any virus produced is collected from these cells and used to re-infect a new GHOST cell culture [13].

Microscopy and Image Analysis.

All steps were automated using Nikon software. For each well of a 96 well plate, 5 sets of images at 200× are collected where multiple cells per well are observed. Images are collected using three different filter cubes, one to observe red fluorescent protein-minimotif chimera cassette, one to observe GFP produced upon HIV infection and one to observe Hoescht nuclei staining; a phase image is also collected. An edge detection algorithm is used to identify cells for each color and the fluorescence signal intensity. The number of cells is determined from the phase image. Background intensities are determined from 10 wells that are not transfected or infected to identify a threshold; the maximal threshold value observed is used. This threshold is then used to calculate the number of cells per well that are above or below the threshold for each color. The program reports the total number of cells, red cells, green cells, and both red and green labeled cells per well. The Strictly Standardized Mean Difference (SSMD) is used to statistically assess each hit [64]. Averages and standard deviations are calculated for each five-well set.

Construction of Chimeric Decoy Inhibitor Library

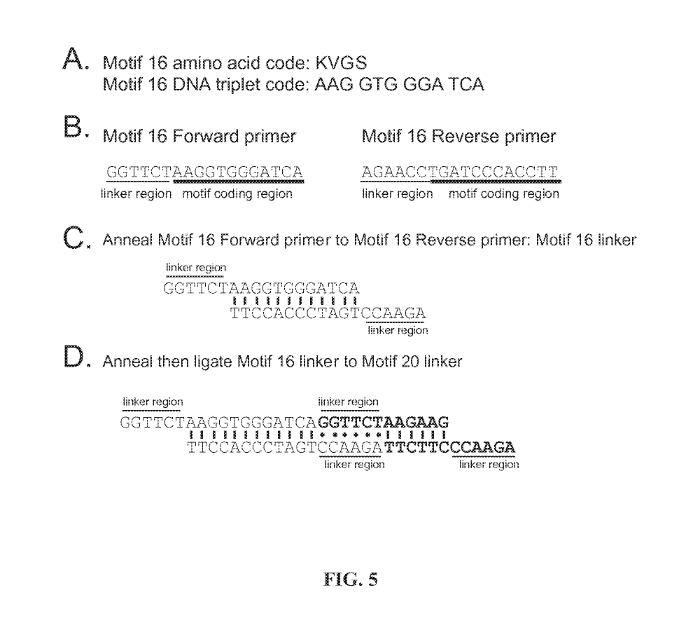

To begin construction of the chimeric decoy inhibitor library, DNA encoding the minimotif s are constructed. The first step in this process is designing the DNA sequences to encode minimotifs in single stranded forms (e.g. minimotif oligonucleotides). A schematic is provided in FIG. 5. The sense and antisense oligonucleotides use the genetic code to encode the minimotif protein, flanked by a “linker” (GGTTCT for forward primer and AGAACC for reverse primer). Each lyophilized primer is resuspended in a volume of autoclaved Milli-Q water to give a concentration of 100 μM. Three microliters of a 100 μM primer are used in a 50 μL phosphorylation reaction containing T4 polynucleotide kinase. The phosphorylation reaction proceeds for 4 hours at 37° C. The kinase is then heat-inactivated at the end of the 4-hr incubation by placing the reaction tubes at 65° C. for 20 minutes. Following heat inactivation, the forward and reverse primers for a given motif are combined into one tube in equimolar amounts. This tube is then incubated at 45° C. for 10 minutes and then slow cooled to room temperature, producing the annealed DNA linker form of the motif.

The annealed DNA linker is viscous and requires a prewarming step at 37° C. for 5 minutes prior to performing downstream applications. Following prewarming, the motif linkers are pooled with the chimeric minimotif decoy initiator and terminator linkers in a 1:1:0.5 ratio. A program on a thermocycler is used to anneal the linkers. The program is as follows: 45° C. for 10 min followed by a 1° C./30 sec decrease until 24° C. is reached, then a 2° C./30 sec decrease until 4° C. is reached. This pool of linkers is then used (8 μL) in a 20 μL ligation reaction using T4 ligase. The ligation reaction proceeds for approximately 4 hours at 16° C. Following ligation, the ligated linker pool is size fractionated using a nick column.

The nick column is first allowed to drain completely of TE buffer. The ligated linker pool is then applied to the nick column membrane. One milliliter of 1×TE buffer is slowly added to the column. Each drop that emerges from the column (˜100 μL/drop) is collected in an individual 1.5 mL tube and labeled as a fraction of the pool. Select pool fractions are amplified using PCR to produce inserts for ligation.

Depending on the size of the ligated pool, a range of fractions from the pool may need to be tested initially to determine the best template for PCR. A forward primer containing a SalI site and a matching sequence to the initiator sequence is paired with a reverse primer containing a BamHI site and a complementary sequence to the chimeric minimotif decoy terminator sequence in the PCR. Thirteen microliters of a fraction are used in a PCR. Following PCR amplification, the PCRs are run on a low melting 1% 1×TAE gel for visualization. Once DNA bands are confirmed, these bands are excised from the gel to then undergo nucleic acid/gel purification using a gel purification kit. The purified DNAs (e.g. inserts) are then subjected to a BamHI/SalI restriction digest to produce compatible 5′ and 3′ ends for future ligation reactions into the mcherry plasmid. The BamHI/SalI digests proceed for approximately 1 hr and then undergo phenol/chloroform extraction twice to remove the restriction digest enzymes. The digested insert samples are then precipitated to concentrate the DNA into a smaller volume. Following concentration, the DNA is now ready to be used in ligation reactions.

The insert DNA is ligated into the BamHI/SalI/phosphatase-treated pRSET.mcherry vector in a 3:1 ratio. The ligation fuses the insert to the end of the coding region for red fluorescent protein. The total volume of the ligation reaction is 11 μL. The ligation proceeds for 30 minutes and is followed by transformation of the reaction into 90 μL of competent E. coli cells. The transformation takes place on ice for 30 minutes. The cells are then heat shocked at 42° C. for 30 seconds followed by an ice incubation step for 5-10 minutes. Two hundred microliters of Luria Broth is added to the cells, which are then placed in a 37° C. shaking incubator for one hour. Following the 1 hour incubation, 250 μL of cells are plated on a LB-kanamycin plate and then incubated overnight at 37° C.

The next day, colonies from the LB-kanamycin plate are inoculated into 2 mL LB-kanamycin cultures and incubated overnight in a 37° C. shaking incubator. The following morning, minipreps are performed to purify the DNA chimeric motif plasmids from the LB-kanamycin cultures. These DNAs are then tested for presence of minimotif chimera cassettes. A 1 hour SalI/BamHI restriction digest is performed on 10 μL of miniprep DNA followed by visualization on a 1% 1×TAE agarose gel. If an insert larger than the combination of initiator+terminator sequence is present, the clone is considered “good” and can be used in downstream transfection experiments.

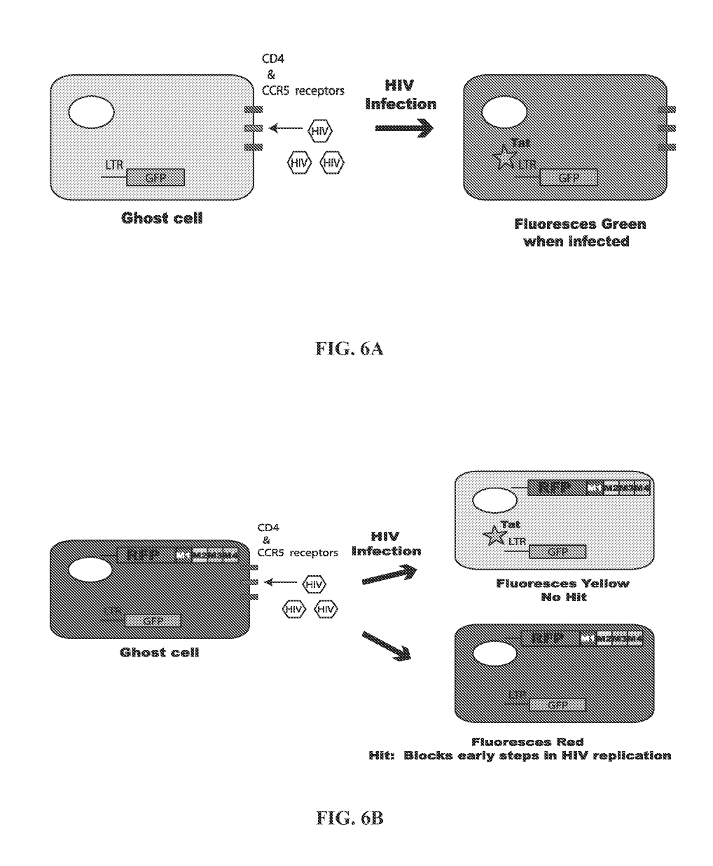

Good clones are used in transfection of a reporter mammalian cell line, Ghost (3) Hi-5. The Ghost (3) Hi-5 cell line is “derived from HOS cells. Stably transduced with MV7neo-T4 retroviral vector, and stably cotransfected with the HIV-2 LTR driving GFP expression and the CMV IE driving hygromycin-resistance.” Infection by a functional HIV particle will cause these cells to produce green fluorescent protein (GFP) as depicted in FIG. 6A. This is the result of the HIV Tat protein inducing production of GFP.

The basic premise of the fluorescence screen is depicted in FIG. 6B. 100 ng of CMD clone DNA is transfected into 5000 Ghost (3) Hi-5 cells. Those cells that take up the DNA will then be able to make the red fluorescent protein-random minimotifs chimeric protein. This will cause the cell to glow “red”. Once red cells have emerged (24 hrs post transfection), we challenge these cells with HIV. If HIV can successfully enter and perform the first steps of the replication cycle, green fluorescent protein will be made. The presence of both green and red fluorescent protein will cause the cell to appear yellow when both signals are overlaid. This constitutes a negative result. A positive result is when the cells remain only red, even in the presence of HIV.

Cells are imaged using a microscope with the necessary filters to capture FITC (green fluorescent protein) and TRITC (red fluorescent protein) signal.

### Examples of Chimeric Minimotif Decoy Initiators

I1SalIBiotFor
(SEQ ID NO: 1)
[Btn]TCGACGGAGCA
I1SalIRev
(SEQ ID NO: 2)
GCCTCGTCCAAGA
I1ReverseA
(SEQ ID NO: 3)
AGAACCTATTCTTGCTCCG
I1ReverseB
(SEQ ID NO: 4)
AGAACCTCGTATTCTTGCTCCG
I1ReverseC
(SEQ ID NO: 5)
AGAACCTACGGTTCTTGCTCCG
(SEQ ID NO: 1)
B-TCGACGGAGCA
(SEQ ID NO: 6)
GCCTCGTCCAAGA
(SEQ ID NO: 3)
B-TCGACGGAGCA
GCCTCGTTCTTATCCAAGA
(SEQ ID NO: 4)
B-TCGACGGAGCA
GCCTCGTTCTTATGCTCCAAGA
(SEQ ID NO: 5)
B-TCGACGGAGCA
GCCTCGTTCTTGGCATCCAAGA

### Examples of Primer Patches

I1ApatchFor
AGAATA
I1BpatchFor
AGAATACGA
I1CpatchFor
AGAACCGTA

### Examples of Chimeric Minimotif Decoy Terminators

T1MycFor
(SEQ ID NO: 7)
GGTTCTATGGCATCAATGCAGAAGCTGATCTCAGAGGAGGACCTGTGAG
T1MycRev
(SEQ ID NO: 8)
GGATCCTCACAGGTCCTCCTCTGAGATCAGCTTCTGCATTGATGCCAT

### REFERENCES

1. Balla S, Thapar V, Luong T., Faghri T, Huang C H, Rajasekaran S, del Campo J J, Shin J H, Mohler W A, Maciejewski M W, Gryk M, Piccirillo B, Schiller S R & Schiller M R (2006) Minimotif Miner, a tool for investigating protein function. NatMethods 3, 175-177.
2. Rajasekaran S, Balla S, Gradie P, Gryk M R, Kadaveru K, Kundeti V, Maciejewski M W, Mi T, Rubino N, Vyas J & Schiller M R (2009) Minimotif miner 2nd release: a database and web system for motif search. Nucleic Acids Res. 37, D185-D190.
3. Mi T, Merlin J C, Deverasetty S, Gryk M R, Bill T J, Brooks A W, Lee L Y, Rathnayake V, Ross C A, Sargeant D P, Strong C L, Watts P, Rajasekaran S & Schiller M R (2012) Minimotif Miner 3.0: database expansion and significantly improved reduction of false-positive predictions from consensus sequences. Nucleic Acids Res. 40, D252-260.
4. Ott J, Kamatani Y & Lathrop M (2011) Family-based designs for genome-wide association studies. Nat. Rev. Genet. 12, 465-474.
5 Merlin J C, Rajasekaran S, Mi T & Schiller M R (2012) Reducing false-positive prediction of minimotifs with a genetic interaction filter. PLoS ONE 7, e32630.
6. Mi T, Rajasekaran S, Merlin J C, Gryk M & Schiller M R (2012) Achieving High Accuracy Prediction of Minimotifs. PLoS ONE 7, e45589.
7. Rajasekaran S, Merlin J C, Kundeti V, Mi T, Oommen A, Vyas J, Alaniz I, Chung K, Chowdhury F, Deverasatty S, Irvey T M, Lacambacal D, Lara D, Panchangam S, Rathnayake V, Watts P & Schiller M R (2010) A computational tool for identifying minimotifs in protein-protein interactions and improving the accuracy of minimotif predictions. Proteins 79, 153-164.
8. Rajasekaran S, Mi T, Merlin J C, Oommen A, Gradie P & Schiller M R (2010) Partitioning of minimotifs based on function with improved prediction accuracy. PLoS ONE 5, e12276.
9. Sargeant D P, Gryk M R, Maciejewski M W, Thapar V, Kundeti V, Rajasekaran S, Romero P, Dunker A K, Li S S-C, Kaneko T & Schiller M R (2012) Secondary structure, a missing component of sequence-based minimotif definitions. PloS One in press.
10. Vyas J, Nowling R J, Maciejewski M W, Rajasekaran S, Gryk M R & Schiller M R (2009) A proposed syntax for Minimotif Semantics, version 1. BMC Genomics 10, 360.
11. Kok K-H, Lei T & Jin D-Y (2009) siRNA and shRNA screens advance key understanding of host factors required for HIV-1 replication. Retrovirology 6, 78.
12. Zhou H, Xu M, Huang Q, Gates A T, Zhang X D, Castle J C, Stec E, Ferrer M, Strulovici B, Hazuda D J & Espeseth A S (2008) Genome-scale RNAi screen for host factors required for HIV replication. Cell Host Microbe 4, 495-504.
13. Brass A L, Dykxhoorn D M, Benita Y, Yan N, Engelman A, Xavier R J, Lieberman J & Elledge S J (2008) Identification of Host Proteins Required for HIV Infection Through a Functional Genomic Screen. Science 319, 921-926.
14. Friedrich B M, Dziuba N, Li G, Endsley M A, Murray J L & Ferguson M R (2011) Host factors mediating HIV-1 replication. Virus Res. 161, 101-114.
15. Fu W, Sanders-Beer B E, Katz K S, Maglott D R, Pruitt K D & Ptak R G (2009) Human immunodeficiency virus type 1, human protein interaction database at NCBI. Nucleic Acids Res. 37, D417-422.
16. Jager S, Cimermancic P, Gulbahce N, Johnson J R, McGovern K E, Clarke S C, Shales M, Mercenne G, Pache L, Li K, Hernandez H, Jang G M, Roth S L, Akiva E, Marlett J, Stephens M, D'Orso I, Fernandes J, Fahey M, Mahon C, O'Donoghue A J, Todorovic A, Morris J H, Maltby D A, Alber T, Cagney G, Bushman F D, Young J A, Chanda S K, Sundquist W I, Kortemme T, Hernandez R D, Craik C S, Burlingame A, SalI A, Frankel A D & Krogan N J (2012) Global landscape of HIV-human protein complexes. Nature 481, 365-370.
17. König R, Zhou Y, Elleder D, Diamond T L, Bonamy G M C, Irelan J T, Chiang C-Y, Tu B P, De Jesus P D, Lilley C E, Seidel S, Opaluch A M, Caldwell J S, Weitzman M D, Kuhen K L, Bandyopadhyay S, Ideker T, Orth A P, Miraglia L J, Bushman F D, Young J A & Chanda S K (2008) Global analysis of host-pathogen interactions that regulate early-stage HIV-1 replication. Cell 135, 49-60.
18. Kadaveru K, Vyas J & Schiller M R (2008) Viral infection and human disease-insights from minimotifs. Front Biosci. 13, 6455-6471.
19. Agbottah E, Zhang N, Dadgar S, Pumfery A, Wade J D, Zeng C & Kashanchi F (2006) Inhibition of HIV-1 virus replication using small soluble Tat peptides. Virology 345, 373-389.
20. Ammosova T, Berro R, Jerebtsova M, Jackson A, Charles S, Klase Z, Southerland W, Gordeuk V R, Kashanchi F & Nekhai S (2006) Phosphorylation of HIV-1 Tat by CDK2 in HIV-1 transcription. Retrovirology 3, 78.
21. Batonick M, Favre M, Boge M, Spearman P, Honing S &Thali M (2005) Interaction of HIV-1 Gag with the clathrin-associated adaptor AP-2. Virology 342, 190-200.
22. Blot G, Janvier K, Le Panse S, Benarous R & Berlioz-Torrent C (2003) Targeting of the human immunodeficiency virus type 1 envelope to the trans-Golgi network through binding to TIP47 is required for env incorporation into virions and infectivity. J. Virol. 77, 6931-6945.
23. Bryant M & Ratner L (1990) Myristoylation-dependent replication and assembly of human immunodeficiency virus 1. Proc. Natl. Acad. Sci. U.S.A. 87, 523-527.
24. Byland R, Vance P J, Hoxie J A & Marsh M (2007) A conserved dileucine motif mediates clathrin and AP-2-dependent endocytosis of the HIV-1 envelope protein. Mol. Biol. Cell 18, 414-425.
25. Cereseto A, Manganaro L, Gutierrez M I, Terreni M, Fittipaldi A, Lusic M, Marcello A & Giacca M (2005) Acetylation of HIV-1 integrase by p300 regulates viral integration. EMBO J. 24, 3070-3081.
26 Chang D-K & Hsu C-S(2007) Biophysical evidence of two docking sites of the carboxyl heptad repeat region within the amino heptad repeat region of gp41 of human immunodeficiency virus type 1. Antiviral Res. 74, 51-58.
27. De Soultrait V R, Caumont A, Parissi V, Morellet N, Ventura M, Lenoir C, Litvak S, Fournier M & Roques B (2002) A novel short peptide is a specific inhibitor of the human immunodeficiency virus type 1 integrase. J. Mol. Biol. 318, 45-58.
28. Dietz J, Koch J, Kaur A, Raja C, Stein S, Grez M, Pustowka A, Mensch S, Ferner J, Möller L, Bannert N, Tampé R, Divita G, Mély Y, Schwalbe H & Dietrich U (2008) Inhibition of HIV-1 by a peptide ligand of the genomic RNA packaging signal Psi. ChemMedChem 3, 749-755.
29. Donahue J P, Vetter M L, Mukhtar N A & D'Aquila R T (2008) The HIV-1 Vif PPLP motif is necessary for human APOBEC3G binding and degradation. Virology 377, 49-53.
30. Dupont S, Sharova N, DéHoratius C, Virbasius C M, Zhu X, Bukrinskaya A G, Stevenson M & Green M R (1999) A novel nuclear export activity in HIV-1 matrix protein required for viral replication. Nature 402, 681-685.
31. Gautier V W, Sheehy N, Duffy M, Hashimoto K & Hall W W (2005) Direct interaction of the human I-mfa domain-containing protein, HIC, with HIV-1 Tat results in cytoplasmic sequestration and control of Tat activity. Proc. Natl. Acad. Sci. U.S.A. 102, 16362-16367.
32. Hovanessian A G, Briand J-P, Said E A, Svab J, Ferris S, Dali H, Muller S, Desgranges C & Krust B (2004) The caveolin-1 binding domain of HIV-1 glycoprotein gp41 is an efficient B cell epitope vaccine candidate against virus infection. Immunity 21, 617-627.
33. Huang J-H, Lu L, Lu H, Chen X, Jiang S & Chen Y-H [2007) Identification of the HIV-1 gp41 core-binding motif in the scaffolding domain of caveolin-1. J. Biol. Chem. 282, 6143-6152.
34. Huang J-H, Qi Z, Wu F, Kotula L, Jiang S & Chen Y-H (2008) Interaction of HIV-1 gp41 core with NPF motif in Epsin: implication in endocytosis of HIV. J. Biol Chem. 283, 14994-15002.
35. Huang J-H, Yang H-W, Liu S, Li J, Jiang S & Chen Y-H (2007) The mechanism by which molecules containing the HIV gp41 core-binding motif HXXNPF inhibit HIV-1 envelope glycoprotein-mediated syncytium formation. Biochem. J. 403, 565-571.
36. Invernizzi C F, Xie B, Richard S & Wainberg M A (2006) PRMT6 diminishes HIV-1 Rev binding to and export of viral RNA. Retrovirology 3, 93.
37. Lama J & Trono D (1998) Human immunodeficiency virus type 1 matrix protein interacts with cellular protein H03. J. Virol. 72, 1671-1676.
38. Li J, Liu Y, Park I-W & He J J (2002) Expression of exogenous Sam68, the 68-kilodalton SRC-associated protein in mitosis, is able to alleviate impaired Rev function in astrocytes. J. Virol. 76, 4526-4535.
39. Li P L, Wang T, Buckley K A, Chenine A-L, Popov S & Ruprecht R M (2005) Phosphorylation of HIV Nef by cAMP-dependent protein kinase. Virology 331, 367-374.
40. Lindwasser O W & Resh M D (2004) Human immunodeficiency virus type 1 Gag contains a dileucine-like motif that regulates association with multivesicular bodies. J. Virol. 78, 6013-6023.
41. Lopez-Verges S, Camus G, Blot G, Beauvoir R, Benarous R & Berlioz-Torrent C (2006) Tail-interacting protein TIP47 is a connector between Gag and Env and is required for Env incorporation into HIV-1 virions. Proc. Natl. Acad. Sci. U.S.A. 103, 14947-14952.
42. Ott D E, Coren L V, Copeland T D, Kane B P, Johnson D G, Sowder R C 2nd, Yoshinaka Y, Oroszlan S, Arthur L O & Henderson L E (1998) Ubiquitin is covalently attached to the p6Gag proteins of human immunodeficiency virus type 1 and simian immunodeficiency virus and to the p12Gag protein of Moloney murine leukemia virus. J. Virol. 72, 2962-2968.
43. Pasquato A, Dettin M, Basak A, Gambaretto R, Tonin L, Seidah N G & Di Bello C (2007) Heparin enhances the furin cleavage of HIV-1 gp160 peptides. FEBS Lett. 581, 5807-5813.
44. Rousso I, Mixon M B, Chen B K & Kim P S (2000) Palmitoylation of the HIV-1 envelope glycoprotein is critical for viral infectivity. Proc. Natl. Acad. Sci. U.S.A. 97, 13523-13525.
45. Strack B, Calistri A, Accola M A, Palu G & Gottlinger H G (2000) A role for ubiquitin ligase recruitment in retrovirus release. Proc. Natl. Acad. Sci. U.S.A. 97, 13063-13068.
46. Strack B, Calistri A, Craig S, Popova E & Gottlinger H G (2003) AIP1/ALIX is a binding partner for HIV-1 p6 and EIAV p9 functioning in virus budding. Cell 114, 689-699.
47. Weber I T, Wu J, Adomat J, Harrison R W, Kimmel A R, Wondrak E M & Louis J M (1997) Crystallographic analysis of human immunodeficiency virus 1 protease with an analog of the conserved CA-p2 substrate—interactions with frequently occurring glutamic acid residue at P2′ position of substrates. Eur. J. Biochem. 249, 523-530.
48. Yang X & Gabuzda D (1998) Mitogen-activated protein kinase phosphorylates and regulates the HIV-1 Vif protein. J. Biol. Chem. 273, 29879-29887.
49. Yang X, Goncalves J & Gabuzda D (1996) Phosphorylation of Vif and its role in HIV-1 replication. J. Biol. Chem. 271, 10121-10129.
50. Zeng L, Li J, Muller M, Yan S, Mujtaba S, Pan C, Wang Z & Zhou M-M (2005) Selective small molecules blocking HIV-1 Tat and coactivator PCAF association. J. Am. Chem. Soc. 127, 2376-2377.
51. Cai L & Jiang S (2010) Development of peptide and small-molecule HIV-1 fusion inhibitors that target gp41. ChemMedChem 5, 1813-1824.
52. Sargeant D, Deverasatty S, Luo Y, Baleta A V, Zobrist S, Rathnayake V, Russo J C, Vyas J, Muesing M A & Schiller M R (2011) HIVToolbox, an integrated web application for investigating HIV. PloS One 6, e20122.
53. Vodros D & Fenyo E M (2005) Quantitative evaluation of HIV and SIV co-receptor use with GHOST(3) cell assay. Methods Mol. Biol. 304, 333-342.
54. Hart T K, Truneh A & Bugelski P J (1996) Characterization of CD4-120 activation intermediates during human immunodeficiency virus type 1 syncytium formation. AIDS Res. Hum. Retroviruses 12, 1305-1313.
55. Parthasarathi L, Casey F, Stein A, Aloy P & Shields D C (2008) Approved drug mimics of short peptide ligands from protein interaction motifs. J Chem Inf Model 48, 1943-1948.
56. Lindman S, Hernandez-Garcia A, Szczepankiewicz O, Frohm B & Linse S (2010) In vivo protein stabilization based on fragment complementation and a split GFP system. Proc. Natl. Acad. Sci. U.S.A. 107, 19826-19831.
57. Ashburner M, Ball C A, Blake J A, Botstein D, Butler H, Cherry J M, Davis A P, Dolinski K, Dwight S S, Eppig J T, Harris M A, Hill D P, Issel-Tarver L, Kasarskis A, Lewis S, Matese J C, Richardson J E, Ringwald M, Rubin G M & Sherlock G (2000) Gene ontology: tool for the unification of biology. The Gene Ontology Consortium. Nat. Genet 25, 25-29.
58. Vyas J, Nowling R J, Meusburger T, Sargeant D, Kadaveru K, Gryk M R, Kundeti V, Rajasekaran S & Schiller M R (2010) MimoSA: a system for minimotif annotation. BMC Bioinformatics 11, 328.
59. Vyas J, Gryk M R & Schiller M R (2009) VENN, a tool for titrating sequence conservation onto protein structures. Nucleic Acids Res 37, e124.
60. Schiller M R, Mi T, Merlin J C, Deverasetty S, Gryk M R, Bill T J, Brooks A W, Lee L Y, Rathnayake V, Ross C A, Sargeant D P, Strong C L, Watts P & Rajasekaran S (2011) Minimotif Miner 3.0: database expansion and significantly improved reduction of false-positive predictions from consensus sequences. Nucleic Acids Research.
61. Gradie P R, Litster M, Thomas R, Vyas J & Schiller M R (2011) SciReader enables reading of medical content with instantaneous definitions. BMC Med Inform Decis Mak 11, 4.
62. Xu X Y, Joh H D, Pin S, Schiller N I, Prange C, Burger P C & Schiller M R (2001) Expression of multiple larger-sized transcripts for several genes in oligodendrogliomas: potential markers for glioma subtype. Cancer Letters 171, 67-77.
63. Schiller M R, Mains R E & Eipper B A (1997) A novel neuroendocrine signaling pathway. Mol. Endocrinol. 11, 1846-1857.
64. Zhang, Chung & Oldenburg (1999) A Simple Statistical Parameter for Use in Evaluation and Validation of High Throughput Screening Assays. J. Biomol Screen 4, 67-73.

1. A method of preparing a CMD clone comprising,
(a) ligating a chimeric minimotif decoy initiator to a beginning end of minimotif duplex,
(b) ligating a chimeric minimotif decoy terminator to a terminal end of a minimotif duplex thereby forming a minimotif chimera cassette,
(c) ligating the minimotif chimera cassette to an expression vector, wherein the expression vector comprises a promoter and reporter protein under the control of the promoter, wherein the minimotif chimera cassette is ligated in frame with the reporter protein of the expression vector and expression of the minimotif chimera is under the control of the promoter,
thereby preparing a CMD clone.

2. The method of claim 1 wherein the minimotif duplex comprises one or more motif coding regions.

3. The method of claim 1, wherein the minimotif duplex comprises a DNA sequence with a single strand overhang on the 5′ end of one strand that is complementary to a portion of a 3′ strand of a chimeric minimotif decoy initiator; wherein the minimotif duplex comprises a DNA sequence with a single strand overhang on the 3′ end of one strand that is complementary to a portion of a 5′ strand of a chimeric minimotif decoy terminator.

4. The method of claim 3, wherein the DNA overhang comprises 3, 6, 9, 12, 15, 18 or 21 base pairs.

5. (canceled)

6. The method of claim 5, wherein the DNA overhang on the 5′ end of each strand of the minimotif duplex comprises a linker region capable of linking two minimotif chimeras together.

7. The method of claim 1, wherein the chimeric minimotif decoy initiator comprises a Kozak sequence.

8. The method of claim 1, wherein the chimeric minimotif decoy initiator comprises a start codon.

9. The method of claim 1, wherein the chimeric minimotif decoy initiator comprises a restriction cleavage site on the 5′ end.

10. (canceled)

11. (canceled)

12. The method of claim 1, wherein the chimeric minimotif decoy terminator is designed to be ligated onto the 3′ end of the section of one or more minimotifs.

13. The method of claim 1, wherein the chimeric minimotif decoy terminator comprises a protein tag.

14. The method of claim 1, wherein the chimeric minimotif decoy terminator comprises a stop codon.

15. The method of claim 1, wherein the chimeric minimotif decoy terminator comprises a restriction cleavage site.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A method for preparing a chimeric minimotif, comprising
a. introducing a 5′ tagged chimeric minimotif decoy initiator to one or more minimotif chimeras forming a first mixture,
b. ligating the 5′ tagged chimeric minimotif decoy initiator to a beginning end of a minimotif chimera to form a first 5′ tagged initiator minimotif chimera,
c. ligating the first 5′ tagged initiator minimotif chimera with an oligonucleotide patch,
d. purifying the ligated complex of step (c) using the 5′ tag of the 5′ tagged chimeric minimotif decoy initiator of step (a),
e. ligating the 5′ tagged chimeric minimotif decoy initiator to the other end of the minimotif chimera to form a second 5′ tagged initiator minimotif chimera,
f. purifying the ligated complex of step (e) using the 5′ tag of the 5′ tagged chimeric minimotif decoy initiator of step (e).

31. The method of claim 30, further comprising (g) fractionating by size the purified ligated complex of step (f).

32. The method of claim 31, further comprising (h) amplifying select pool fractions using PCR to produce inserts for ligation.

33. The method of claim 32 further comprising (i) visualizing the amplified fractions of step (h), and (j) confirming DNA bands and excising them from the gel to undergo nucleic acid/gel purification.

34. The method of claim 30, wherein the 5′ tagged chimeric minimotif decoy initiator forms an internal duplex.

35. The method of claim 30, wherein after step (a), but prior or during step (b) the first mixture is heated to separate the internal duplex of the 5′ tagged chimeric minimotif decoy initiator.

36. The method of claim 30, wherein the first mixture is cooled after step (b) to allow any unligated 5′ tagged chimeric minimotif decoy initiators to reform an internal duplex.

37. (canceled)

38. (canceled)

39. (canceled)

40. (canceled)

41. A method of preparing a minimotif chimeria cassette, comprising introducing a 5′ tagged chimeric minimotif decoy initiator to one or more minimotif oligonucleotides forming a first mixture, ligating a 5′ tagged chimeric minimotif decoy initiator to a beginning end of a minimotif oligonucleotide to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif oligonucleotide to form a 5′ and optionally 3′ tagged minimotif chimera cassette.

42. A methods of preparing a minimotif chimeria cassette, comprising introducing a 5′ tagged chimeric minimotif decoy initiator to one or more minimotif duplexes forming a first mixture, ligating a 5′ tagged chimeric minimotif decoy initiator to a beginning end of a minimotif duplex to form a first 5′ tagged initiator minimotif chimera, complex purifying the 5′ tagged initiator minimotif chimera, complex using the 5′ tag of the 5′ tagged chimeric minimotif decoy initiator, ligating an optionally 3′ tagged chimeric minimotif decoy terminator to the other end of the minimotif duplex to form a 5′ and optionally 3′ tagged minimotif chimera cassette


SEQUENCE LISTING (8 sequences)

SEQ ID NO: 1 - DNA, 11 bp, Artificial Sequence
  synthetic peptide; primer
tcgacggagca

SEQ ID NO: 2 - DNA, 13 bp, Artificial Sequence
  synthetic construct; primer
gcctcgtccaaga

SEQ ID NO: 3 - DNA, 19 bp, Artificial Sequence
  synthetic construct; primer
agaacctattcttgctccg

SEQ ID NO: 4 - DNA, 22 bp, Artificial Sequence
  synthetic construct; primer
agaacctcgtattcttgctccg

SEQ ID NO: 5 - DNA, 22 bp, Artificial Sequence
  synthetic construct; primer
agaacctacggttcttgctccg

SEQ ID NO: 6 - DNA, 13 bp, Artificial Sequence
  synthetic construct; primer
agaacctgctccg

SEQ ID NO: 7 - DNA, 49 bp, Artificial Sequence
  synthetic construct; primer
ggttctatggcatcaatgcagaagctgatctcagaggaggacctgtgag

SEQ ID NO: 8 - DNA, 48 bp, Artificial Sequence
  synthetic construct; primer
ggatcctcacaggtcctcctctgagatcagcttctgcattgatgccat